(19) United States
(12) Patent Application Publication
(10) Pub. No.: US 2025/0061568 A1
(43) Pub. Date: Feb. 20, 2025

(54) SSYSTEM AND METHOD FOR DETERMINING CUSTOMIZED VIEWING WINDOWS FOR MEDICAL IMAGES

(71) Applicant: KONINKLIJKE PHILIPS N.V., EINDHOVEN (NL)

(72) Inventors: NICK FLAESCHNER, HAMBURG (DE); FABIAN WENZEL, HAMBURG (DE); ARNE EWALD, HAMBURG (DE); ELIZA TEODORA ORASANU, HAMBURG (DE)

(21) Appl. No.: 18/719,327

(22) PCT Filed: Dec. 16, 2022
(86) PCT No.: PCT/EP2022/086281

§ 371 Date: Jun. 13, 2024

Related U.S. Application Data
(60) Provisional application No. 63/293,108, filed on Dec. 23, 2021.

(51) Int. Cl. G06T 7/00; G06T 7/10; G06V 10/25; G06V 10/60; G16H 30/40

(52) U.S. Cl. CPC G06T 7/0012; G06T 7/10; G06V 10/25; G06V 10/60; G16H 30/40; G06T 2207/30016

(57) ABSTRACT

A method for processing medical image information includes generating a first intensity distribution for a first image region, generating a second intensity distribution for a second image region, calculating values based on the first and second intensity distributions, and automatically determining a custom viewing window based on the calculated values. The custom viewing window is determined to display the first image region and the second image region, which may be in the same image or different images. The images may include brain scan images or other types of images.

Example embodiments disclosed herein relate to processing medical image information.

### BACKGROUND

When a medical image is displayed on a screen, a mapping from the voxel values to grayscale values (e.g., brightness) may be chosen. Typically, the human eye can perceive far fewer numbers of grayscale values (e.g., a maximum of 100) than are contained in a medical image. For example, a 12-bit CT can contain 4096 different voxel values. As a consequence, for different diagnostic tasks, different standard mappings (or viewing windows) with pre-defined parameters have been established.

However, for some tasks, very subtle differences between certain areas (or regions of interest) within one scan or image slice (or between different scans or image slices) should be assessed. In order to make this assessment, a manual operation is performed to select (or guess) which one of the standard viewing windows might be able to make the subtle differences visible to a radiologist. Tuning the viewing window manually is time-consuming and subject to error. For example, important clinical findings are often missed because none of the standard viewing windows are sufficient to reveal those findings, nor are those windows customized in any way to the particular patient to be evaluated.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for processing medical image information, including: generating a first intensity distribution for a first image region; generating a second intensity distribution for a second image region; calculating values based on the first and second intensity distributions; and automatically determining a custom viewing window based on the calculated values, wherein the custom viewing window is determined to display the first image region and the second image region.

Various embodiments are described, wherein determining the custom viewing window includes determining one or more parameters of the custom viewing window.

Various embodiments are described, wherein determining the one or more parameters includes computing the one or more parameters based on difference values calculated between the first and second intensity distributions.

Various embodiments are described, wherein determining the one or more parameters includes applying at least one rule to the difference values to determine the one or more parameters.

Various embodiments are described, wherein the one or more parameters of the custom viewing window increase recognition of differences between the first and second image regions over a viewing window used to display the first and second image regions using one or more other parameters.

Various embodiments are described, wherein the one or more parameters includes at least one of a width or a level of the custom viewing window.

Various embodiments are described, wherein the width is based upon a maximum intensity value in the difference values for which the difference value is non-zero and a minimum intensity value in the difference values for which the difference value is non-zero.

Various embodiments are described, wherein the values calculated based on the first and second intensity distributions include difference values between the first and second intensity distributions.

Various embodiments are described, wherein the first image region and the second image region are in a same image.

Various embodiments are described, wherein: the first image region is a first area in a first brain scan image; the second image region is a second area in the first brain scan image.

Various embodiments are described, wherein: the first image region is derived from a first bounding box; and the second image region is derived from a second bounding box.

Various embodiments are described, further including: automatically determining the first and second image regions based on a segmentation algorithm.

Various embodiments are described, further including: automatically determining the second image region based on a designation of the first image region.

Further various embodiments relate to a medical image analyzer, including: a generator configured to generate a first intensity distribution for a first image region and a second intensity distribution for a second image region; computing logic configured to calculate values based on the first intensity distribution and the second intensity distribution; and a rules engine configured to automatically determine a custom viewing window based on the calculated values, wherein the custom viewing window is determined to display the first image region and the second image region.

Various embodiments are described, wherein the rules engine is configured to determine one or more parameters of the custom viewing window.

Various embodiments are described, wherein determining the one or more parameters includes computing the one or more parameters based on difference values calculated between the first and second intensity distributions.

Various embodiments are described, wherein the rules engine is configured to apply at least one rule to the difference values to determine the one or more parameters.

Various embodiments are described, wherein the one or more parameters includes at least one of a width or a level of the custom viewing window.

Various embodiments are described, wherein the width is based upon a maximum intensity value in the difference values, a minimum intensity value in the difference values, and a scalar.

Various embodiments are described, wherein the first image region and the second image region are in a same image.

### BRIEF DESCRIPTION OF THE DRAWINGS

Additional objects and features of the invention will be more readily apparent from the following detailed description and appended claims when taken in conjunction with the drawings. Although several example embodiments are illustrated and described, like reference numerals identify like parts in each of the figures, in which:

FIG. 1 illustrates an embodiment of a medical image analyzer.

FIG. 2 illustrates an embodiment of a method for automatically determining a custom viewing window for displaying medical images.

FIGS. 3A and 3B illustrate example images of brain scans.

FIG. 4A illustrates an example of generating a difference histogram in accordance with one embodiment, and FIG. 4B illustrates an example of a difference histogram not generated as a curve.

FIG. 5A illustrates a brain scan image displayed using a standard viewing window, and FIG. 5B illustrates a brain scan image displayed using an embodiment of a custom viewing window.

FIG. 6 illustrates an embodiment of a medical image analyzer.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

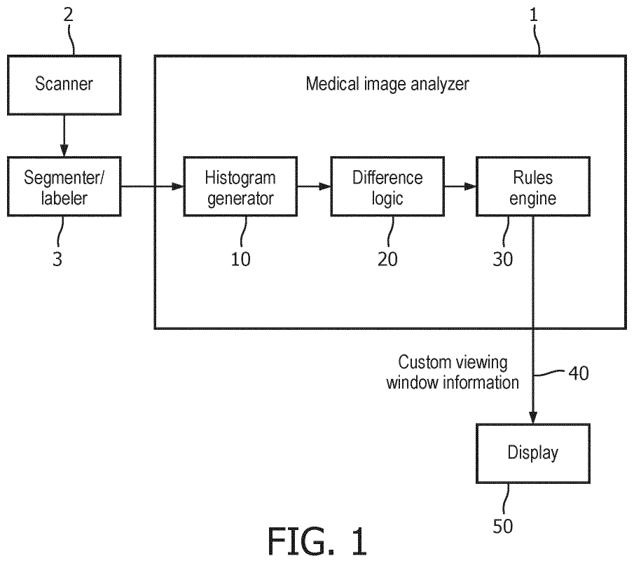

FIG. 1 illustrates a system for automatically determining a custom viewing window for displaying medical images. The system may include a medical image analyzer 1 for processing medical images derived from a computed tomography (CT) scanner or another type of medical imaging system. The example of CT images will be discussed in connection with the following embodiments.

The medical image analyzer 1 may be coupled between a medical image scanner 2 and a display 50. As indicated above, the scanner may be various types of scanners including but not limited to a CT scanner and a medical resonance imaging (MRI) scanner. In one embodiment, a segmenter and/or labeler 3 may be coupled between (or included in) the medical image analyzer for pre-processing images output from the scanner. The segmenter may, for example, automatically segment an image into various regions of interest or may allow for the designation of bounding boxes to designation regions of interest. Automatic segmentation may be performed, for example, to identify Alberta Stroke Program Early (non-contrast) CT ASPECTS regions in an image slice of the brain. In such a case, the labeler may insert contours around and label the various ASPECTS regions in the image slice for further analysis.

Referring to FIG. 1, the medical image analyzer includes an intensity distribution generator 10, computing logic 20, and a rules engine 30. The intensity distribution generator may generate an intensity distribution of a first image region and a second image region corresponding to the images output from the scanner. The first image region and the second image region may be in the same image (e.g., when left and right ASPECTS regions are to be considered for analysis) or may be in different regions (e.g., when brain scans of a same patient are to be analyzed at different times). The first image region may be determined, for example, based on bounding boxes overlaid on the image(s), may be determined automatically by the segmenter, or may be determined using a different technique. In some cases, more than two regions of interest may be analyzed as described in greater detail below.

In one embodiment, the intensity distribution generator may be a histogram generator 10. For illustrative purposes, the intensity distribution generator will be assumed to be a histogram generator for the balance of this discussion. In operation, the histogram generator 10 may extract intensity values from each of the regions of interest and generate a first histogram and a second histogram for corresponding ones of the regions of interest. Once generated, the histograms may be further analyzed by the computing logic.

The computing logic 20 may calculate a plurality of values based on the first histogram and the second histogram. In one embodiment, the computing logic 20 may include difference logic that computes difference values based on subtracting the intensity values in the first histogram and the second histogram. For illustrative purposes, the computing logic 20 be assumed to be a histogram generator for the balance of this discussion. A more detailed discussion of how these values are generated is provided below.

The rules engine 30 applies one or more predetermined rules to the difference values output from the difference logic. The rules may be used to generate a custom viewing window which may be used as a basis for displaying the regions of interest in a way that increases recognition of features that otherwise would be hidden if standard viewing windows were used to view the regions. In one embodiment, the rules applied by the rules engine determines one or more parameters of the viewing window. The parameters may include for example one or both of a window width and a window level. Generating the parameters from the difference values allows for customization of the viewing window on a patient-by-patient basis. Once the parameter(s) for the custom viewing window have been generated, information 40 defining the viewing window may be output to a display 50 for viewing by healthcare professionals.

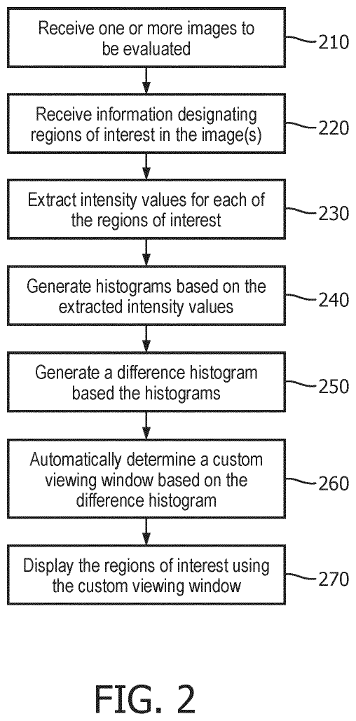

FIG. 2 illustrates operations included in an embodiment of a method for automatically determining a custom viewing window for displaying medical images. The method may be performed, for example, by any of the system (or medical image analyzer) embodiments described herein or may be performed by a different system. For illustrative purposes, the method will be described as being performed by the system of FIG. 1.

Referring to FIG. 2, the method includes, at 210, receiving one or more images to be evaluated. The one or more images may correspond, for example, to a same patient. In such a case, the image(s) may include any portion of the body of the patient determined based on the purpose for which the image(s) were acquired. In one non-limiting embodiment, the patient may be one suspected of having a brain abnormality such as a lesion produced by an ischemic stroke or the abnormality may correspond to another brain condition. In this case, the image generated by the scanning system (which, for example, may be a computed tomography scanner) may be non-contrast CT (NCCT) brain scan images. The CT scanner may be local (e.g., in the same hospital or clinical setting) or remotely located from the processing logic of the system implementing the method. In this latter case, the system and method embodiments may be used in an outsourcing context where, for example, the images are received from a network.

The brain scan image may include an axial slice corresponding to a particular area of the brain where one or more types of brain abnormalities may occur given, for example, a suspected condition of the patient. For example, when the patient is suspected of having suffered an ischemic stroke, the slice corresponding to the brain scan image may include a region where the middle cerebral artery (MCA) is located. As will be described in greater detail, such a stroke will produce an occlusion (e.g., an ischemic lesion) in the image at the location of the MCA, at least with respect to the hemisphere of the brain where the stroke occurred. The image slice may be stored in a memory of the system, with or without normalization, for processing in accordance with the operations described herein. In one embodiment, two or more images may be received for evaluation.

At 220, the system receives information designating at least two regions of interest in the one or more images. When the one or more images include at least one image (e.g., a CT scan), the at least two regions of interest may be determined in various ways. For example, a first region of interest and a second region of interest may be selected to correspond to areas enclosed within respective bounding boxes overlaid on a same image or on different images. The bounding boxes may be drawn, for example, by a radiologist. In another embodiment, the regions of interest may correspond to areas designated by contours or polygons overlaid or drawn on the same or different images. The example where the regions of interest are in the same image will be described for example purposes.

In one embodiment, the regions of interest may correspond to one of a plurality of areas generated, for example, by an image segmentation algorithm. For example, in a brain scan implementation, the segmentation algorithm may automatically identify and segment the image into a plurality of regions defined by the ASPECTS protocol. When segmentation is performed according to the ASPECTS protocol, a total of twenty regions of interest are defined: ten regions on the left hemisphere of the brain and ten regions on the right hemisphere of the brain in two image slices. The ten regions in the left hemisphere may be of the same types as the regions on the right hemisphere, and thus the ten regions on the left hemisphere may be considered to be complementary to respective ones of the regions on the right hemisphere, thereby forming complementary region pairs.

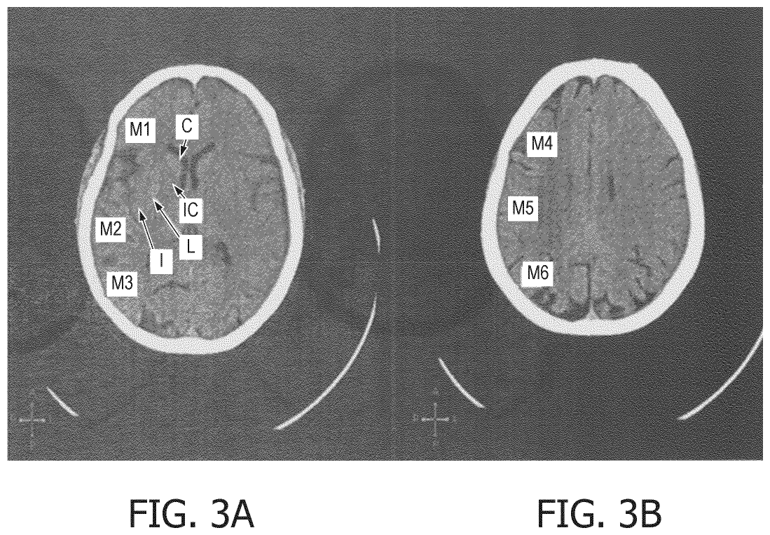

Table 1 identifies the ten regions in each hemisphere of the brain that correspond to the ASPECTS protocol. Seven of the ten regions appear in the superior image slice (FIG. 3A) and the remaining three regions appear in the inferior image slice (FIG. 3B) illustrated on a per-hemisphere basis. Together, all ten regions may be considered candidate regions for lesions or other abnormalities that may adversely affect the brain.

TABLE 1

| Acronym | Region | Acronym | Region |
|---|---|---|---|
| C | Caudate head | M2 | MCA cortex lateral to |
| | | | insular ribbon, |
| | | | corresponding to anterior |
| | | | temporal lobe |
| IC | Internal Capsule | M3 | posterior MCA cortex, |
| | | | corresponding to |
| | | | posterior temporal lobe |
| L | Lentiform Nucleus | M4 | anterior MCA territory, |
| | | | immediately |
| | | | superior to M1 |
| I | Insular Ribbon | M5 | lateral MCA territory, |
| | | | immediately |
| | | | superior to M2 |
| M1 | anterior MCA cortex, | M6 | posterior MCA territory, |
| | corresponding to | | immediately |
| | frontal operculum | | superior to M3 |

In one embodiment, segmentation of the image slices may be performed automatically in 3D using a model-based approach. The model may process the images to identify (extract) and then generate overlay graphics outlining the contours of each of the ten regions of interest. Then, the model may label each of the segmented regions as illustrated in FIGS. 3A and 3B. The contours may be matched with one another using, for example, multi-planar reformatting. The automatic segmentation of the image slices may be performed, for example, in accordance with the techniques described in WO 2020/109006 or as described in the article entitled “Automatic model-based segmentation of the heart in CT images” by Ecabert O, Peters J, Schramm H, Lorenz C, von Berg J, Walker M J, Vembar M, Olszewski M E, Subramanyan K, Lavi G, Weese J., IEEE Trans Med Imaging, 2008 September; 27(9):1189-201 (which disclose various techniques including, but not limited to, ones that apply shape-constrained anatomical active contour model using a mesh of triangles to patient scans), the contents of which are all incorporated by reference herein.

For a brain-scan implementation as described above, operation 220 may include receiving information designating any two of the ASPECTS regions. For example, the two designated regions may be the same region on complementary sides of the brain or may be different ones of the ASPECTS regions. In either case, the two regions may be designated, for example, by manual selection by a radiologist or other healthcare professional or the algorithm may select the two regions of interest, for example, based on preferences embedded in the control software implementing the algorithm. In one embodiment, selection of one ASPECTS region as a region of interest may automatically trigger (e.g., by control software of the segmenter 3) selection of the second region of interest as the complementary ASPECTS region on the other lateral side of the brain. When a plurality of images are received in operation 210, the regions of interest may be in different ones of the images, for example, in the case where one of the images is a follow-up image.

At 230, intensity values are extracted for each of the regions of interest designated by the information received in operation 220. The intensity values may be expressed in Hounsfield Units (HU), which indicate how strongly radiation is attenuated during scanning. The degree of attenuation (indicated, for example, by CT attenuation coefficients) correlates to tissue density. Thus, the HU values may provide a quantitative indication of the density (and thus the types of tissues) located in the designated regions of interest.

For purposes of image visualization, the HU values may correspond to voxel values expressed on a color scale or grayscale, e.g., various shades between black and white inclusive. Tissues with lower density may have darker shades (or intensity), while tissues with higher density may be expressed with lighter shades (or intensity). In some cases, the HU or voxel values may undergo normalization or scaling, for example, by shifting them by a predetermined constant value. In operation 230, once the HU values have been extracted for each of the two regions of interest, they may be stored in corresponding tables, e.g., a first table may include HU values for the first region of interest and a second table may include HU values for the second region of interest.

At 240, intensity distributions are generated based on the intensity values extracted for the regions of interest. In one embodiment, the intensity distributions may be normalized and generated in the form of histograms based on the HU values extracted in operation 230. For example, a first histogram may be generated for the first region of interest and a second histogram may be generated for the second region of interest. The histograms, thus, represent distributions of all voxels within respective ones of the regions of interest. Each histogram may be constructed so that values along the x-axis correspond to HU values and values along the y-axis correspond to the numbers of voxels having corresponding ones of those values in the region of interest.

In one embodiment relating to brain scans, the intensity histograms may be generated on a bi-lateral basis, where the first region of interest includes an ASPECTS region in the left lateral portion of the brain and the second region of interest includes the same ASPECTS region in right lateral portion of the brain. In this case, the histograms provide an indication of the voxel content in complementary regions of the brain. In generating the histograms, the range of HU values may be the full range of HU values or a predetermined subset of values within the full range which, for example, may be considered relevant to the particular type(s) of brain abnormality of interest.

In one embodiment, the HU values may be optionally binned. In such a case, in each histogram HU value ranges are illustrated on the x-axis and values on the y-axis correspond to the numbers of voxels in that range. In another embodiment, the intensity histograms may have values limited to a range of between an HU value of 10 and an HU value of 60 spread across 25 bins with each bin have a size of 2 HUs. This range may be considered suitable for some applications, e.g., the lower HU boundary value of 10 excludes large parts of the CSF, where the upper HU boundary value of 60 completely includes gray matter but excludes calcifications and hemorrhagic lesions. The histogram data may be set based on a different range of HU values in another embodiment.

Once the histograms have been generated, the values in each histogram may be subject to a normalization operation. This may involve, for example, dividing each y value in each histogram by the total number of voxels to normalize the histogram voxel values to reside between 0 and 1 and so that the sum of all the histogram voxel values is 1.

At 250, derivative histogram data is generated based on the histograms formed in operation 240. The derivative histogram data may include, for example, a difference histogram generated for the first and second regions of interest. For example, the difference histogram may be generated based on a difference between HU values in the histogram of the first region of interest and the HU values in the histogram of the second region of interest. In the case where the two regions of interest are complementary ASPECTS regions of a brain scan image, the difference histogram provides a substantive (quantitative and qualitative) indication of how different the intensity values are in those regions and also characterize the type(s) of tissues and, if present, other artifacts at those locations. This, in turn, may allow for the automatic selection of a viewing window for the image in the manner described in greater detail below.

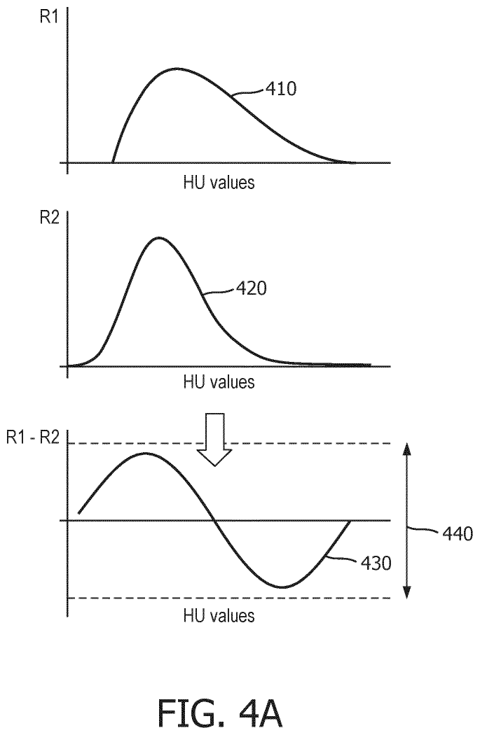

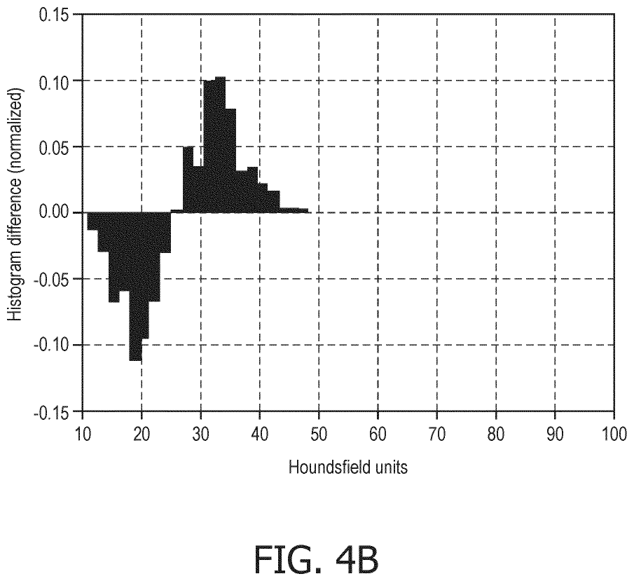

FIG. 4A illustrates an example of a difference histogram (generalized to a curve) 430 which may be generated based on a histogram (generalized to a curve) 410 corresponding to the first region of interest and a histogram (generalized to a curve) 420 corresponding to the second region of interest in the image. For purposes of illustration, histogram 410 is labeled R1 and histogram 420 is labeled R2. Thus, difference histogram 430 may be computed by subtracting intensity values in the first histogram from intensity values in the first reference histogram, e.g., by computing the equation: R1−R2, thus accounting for the negative difference values in histogram 430.

The resulting difference histogram 430 has difference values that fall within a range 440, which may serve as a basis for characterizing the types of tissue or other artifacts in the first and second regions and also may provide a basis for determining window selection. An example difference histogram (not generalized to a curve and) having values normalized to within a predetermined range of difference values on the y-axis relative to HU values on the x-axis is illustrated in FIG. 4B.

At 260, a customized viewing window is automatically determined based on the difference histogram. The customization may involve determining one or more parameters of a viewing window that allow fine differences between the two regions of interest to be recognizable, which differences would not be recognizable if the same regions were displayed using standard viewing windows, e.g., windows having preset parameters typically used in the field displaying CT images. This may be understood in greater detail as follows.

The viewing window for displaying a medical image may be determined by the range of HU values used to display the image. Standard viewing windows are based on a discrete number of preselected ranges of HU values intended to emphasize the display of certain types of tissue, bone, or other physiological features in the area scanned by the CT imager. For example, a standard window known as a “brain window” has a width of 80 HU values, centered at an HU value of 40. When such a viewing window is used, every voxel having an HU value of 0 is displayed as black and every voxel having an HU value above 80 is displayed as white. The HU values within the window width are linearly mapped to grayscale values.

Using such a window limits (or hides) various types of physiological information that may be considered important to diagnosis and treatment. For example, the brain window discussed above may allow for certain types of brain tissue to be displayed, but may hide other types of tissue such as cranial (bone) structure. Thus, using a brain window in cases where cranial bone structure is also of interest excludes information that may be considered valuable important for providing medical care.

Moreover, the use of standard (predefined) viewing windows are not optimally tailored in cases where, for example, subtle differences are to be compared between to regions of interest in an image (e.g., those included in two bounding boxes). For example, in an ASPECTS application where the lentiform nucleus region in the left hemisphere is to be compared with the lentiform nucleus region in the right hemisphere, none of the standard viewing windows may have the parameters for allowing a detailed visual assessment between the two regions. This problem is compounded by how these and other ASPECTS regions may vary significantly from patient to patient. Standard viewing windows do not address this disparity among patients and therefore are ineffective. As a result, the preset HU ranges of the standard viewing windows hide certain important health aspects of the patient that have clinical significance. Additionally, the selection of which standard window to use is presently a manual determination based on the judgment of the radiologist. Such an approach is time-consuming and subject to error.

To solve and these and other problems, in accordance with one or more embodiments a customized range of HU values is automatically determined to display the first and second regions of interest based on the difference histogram generated between those regions. Because the difference histograms are based on values generated for each particular patient, the viewing window may be customized to conform to the specific condition of each patient, which, for example, may improve or optimize use for comparison of ASPECTS regions which are unable to be performed using standard viewing windows.

Thus, for example, a different viewing window may be determined for each specific pair of regions of interest that may not conform to any of the standard viewing windows. This allows features that otherwise would be hidden by standard viewing windows to be displayed in an accurate cognizable way, which, in turn, may allow healthcare professionals to diagnose diseases or other health conditions and/or develop more effective treatment plans.

In determining the viewing window, one custom parameter that may be determined is window width, e.g., the specific range of HU values to be displayed. Window width determines image contrast, e.g., the greater the width, the less the contrast. Another custom parameter that may be determined is window level. Window level determines image brightness, e.g., the greater the level, the greater the brightness. Setting the window level determines the central or midpoint grayscale value for the window width. Setting a custom window level may, for example, allow different types of tissue in the first and second regions to become apparent where they otherwise would be hidden if standard viewing windows were used.

In one embodiment, the customized parameter(s) of the viewing window may be determined based on one or more predetermined rules. The rule(s) may be generated, for example, to reveal features in the first and second regions that would otherwise be hidden if a standard viewing window were used to display the image. The rule(s) therefore serve to customize display of the image in a way that improves (or even optimizes) display and recognition of important clinical information embedded in the intensity values of the image.

When the parameters include width and level, in one embodiment the rules set forth in Equations 1 and 2 may be applied to the values in the difference histogram to compute the width and level of the customized viewing window.

$$Width = max \{ x \vert abs ( DIFF\_A \_B ( x ) ) > 0 \} - min \{ x \vert abs ( DIFF\_A \_B ( x ) ) > 0 \} \qquad ( 1 )$$

$$Level = min \{ x \vert abs ( DIFF\_A \_B ( x ) ) > 0 \} + \frac{1}{2} Width \qquad ( 2 )$$

In Equations 1 and 2, the term A corresponds to the histogram generated for the first region of interest and the term B corresponds to the histogram generated for the second region of interest. One these values have been obtained, the difference histogram DIFF_A_B may be computed by subtracting the histogram values of the first region of interest from corresponding histogram values of the second region of interest. The width calculated by Equation 1 is based upon finding the maximum x value for which the difference value is greater than zero and the minimum x value for which the difference value is that is greater than zero and taking the difference between these values. The midpoint value of the window may then be set based on Equation 2.

In another embodiment, a different rule may be applied to determine the width and level of the custom viewing window. For example, the width may be set based on predetermined quantiles of the values in the difference histogram as indicated in Equation 3 and the level may be computed based on Equation 4:

$$Width = scaler ( DIFF\_A \_B , PERCENTAGE ) \qquad ( 3 )$$

$$Level = min \{ x \vert abs ( DIFF\_A \_B ( x ) ) > 0 \} + \frac{1}{2} Width \qquad ( 4 )$$

where scaler is a function that ensures that a certain PERCENTAGE of the difference range is included. In one example implementation, PERCENTAGE may be set to include 95% (i.e., 0.95) of the range. This value may be preset by the system or allowed to be varied by a user.

In other embodiments, rules different from those indicated above may be used to automatically generate one or more parameters for the custom viewing window. In one embodiment, one or more lookup tables may be stored in system memory for use by the rules engine to compute the parameters of the viewing window. In this case, predetermined sets of values for corresponding difference histograms may be calculated and stored in advance. In such a case, once the values of the difference histogram are known for the first and second regions of a particular image under analysis, the rules engine may perform a search of the lookup table to find the custom width and level of the viewing window.

At 270, the regions of interest (or the full image(s) containing the regions of interest) are displayed based on the custom viewing window automatically determined in operation 260. Because the viewing window is customized to the intensity values generated for a specific patient scan, features that otherwise would be hidden are displayed in recognizable form. For example, differences in grayscale values in the first and second regions of the image become discernible. These differences may allow a physician, radiologist or other healthcare professional to locate problems, abnormalities or other effects that may lead to a more effective course of treatment for the specific condition the patient is experiencing. This customized approach represents a significant improvement in the case where all brain scans are subject to the same small set of standard viewing windows.

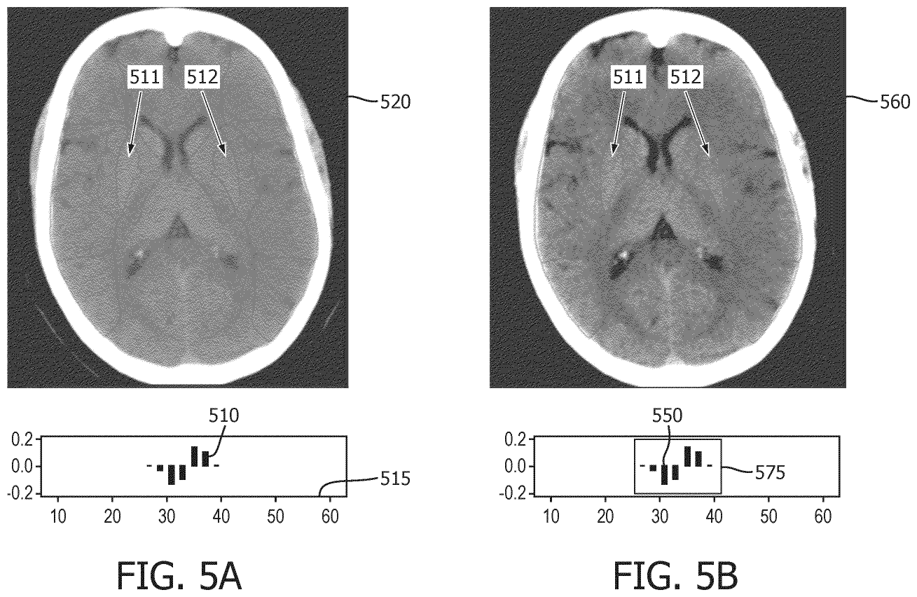

FIG. 5A illustrates an image of a brain scan displayed using a standard viewing window, and FIG. 5B illustrates an image of the same brain scan displayed using a custom viewing window generated in accordance with one or more embodiments. The brain scan is of a patient who has suffered a stroke, and the images have been segmented into ASPECTS regions corresponding to a superior image slice.

In FIG. 5A, a difference histogram 510 is illustrated depicting the intensity differences between regions including the putamens in the left and right hemispheres of the brain. These regions (e.g., corresponding to the left and right lentiform nucleus ASPECTS regions) are labeled with arrows 511 and 512 and are displayed using the standard viewing window. As illustrated in FIG. 5A, the width of the standard viewing window 515 spans a large range of HU values, most of which do not correspond to any of the difference values in the difference histogram, e.g., the width of the standard viewing window is much greater than the range of HU values in the difference histogram. As a result, the grayscale values (and thus the clinical information) in regions 511 and 512 are virtually indistinguishable in the left and right putamen regions.

However, one of the putamen regions 512 includes a lesion caused by the stroke. The existence of this lesion is not apparent in image 520 because of the indistinguishable grayscale values in regions 511 and 512 produced by the standard viewing window. Thus, using the standard viewing window hides clinical information which may be considered important in diagnosing and treating the patient.

In FIG. 5B, a histogram 550 is illustrated depicting the intensity differences between the same putamen regions in the left and right hemispheres of the brain, labeled 511 and 512. However, unlike the case in FIG. 5A, the brain scan image 660 is displayed using custom viewing window generated in accordance with the system and method embodiments described herein. Unlike the standard viewing window 515 applied in FIG. 5A, the custom viewing window 575 has been adjusted to the values in difference histogram 550, e.g., in FIG. 5A, the standard viewing window 515 is centered at an HU value of 35 and has a width that extends from an HU value of 5 to an HU value of 65. In contrast, the custom viewing window 575 of FIG. 5B is centered at HU value of 33 and has a width extending from an HU value of 25 to an HU value of 41 HU and thus conforms to the values in the difference histogram 550 generated from the image.

As is evident from a comparison of FIGS. 5A and 5B, the image results displayed in FIG. 5B using the custom viewing window 575 are very different from the image results displayed using the standard viewing window 515 in FIG. 5A. Using the custom viewing window, the grayscale values in the putamen region 512 (which includes the stroke lesion) is substantially darker than the grayscale values in the putamen region 511. This is a direct result of the customized viewing window and would be clearly recognized by a healthcare professional. What this comparison illustrates is that important clinical findings are hidden by use of the standard viewing window, but these clinical findings are readily apparent when the custom viewing window is used to display the brain scan.

In one embodiment, the first and second regions of interest may be in different images, e.g., the first region of interest may be included in a first brain scan image taken at a first time and the second region of interest may be included in a second brain scan image from the same patient for the same corresponding area at a second subsequent time. As previously indicated, the regions of interest may be manually selected (e.g., via bounding boxes) or automatically generated, for example, by a segmentation algorithm. The difference between the first and second times may be any length of time. Such an implementation may be beneficial, for example, to allow for a determination of whether any changes have occurred over time.

For example, the first brain scan image may correspond to FIG. 5B where a lesion has formed in the putamen region as a result of a stroke. The second brain scan image may be taken during a follow-up exam or during the occurrence of another complication weeks, months, or years later. In such a case, the difference histogram may be generated based on the HU values extracted for the same putamen region of the patient. The difference histogram may then be input into the rules engine to generate one or more parameters of a custom viewing window for determining the condition of the patient. Before the difference histogram is generated, the images (or the regions of interest) may undergo registration to allow for more accurate results to be generated.

In some embodiments, more than one region of interest may be compared. In such a case, three or more regions of interest (for the same or different images) may be grouped into at least two meta-regions of interest, with each meta-region of interest corresponding to a union of at least two of the three or more regions of interest. For example, take the case where three regions of interest are designated. A first meta-region may correspond to the first and second regions of interest, and a second meta-region may correspond to the second and third regions of interest. In some cases, a third meta-region may correspond to the first and third regions.

In such a case, a difference histogram may be calculated for each of the meta-regions based on a difference in the histograms (or voxel values) generated for the first, second, and third regions of interest. Different viewing windows may then be generated by applying one or more rules to the values in the difference histograms, and the custom viewing windows may then be used to display the images as described above. As with all embodiments described herein, instead of histograms, other types of normalized intensity distributions may be generated for generating the parameter(s) of the custom viewing windows.

While CT scans are discussed as an example in some embodiments, the systems and methods described herein may be applied to other types of images in other embodiments, including but not limited to magnetic resonance imaging (MRI) scans.

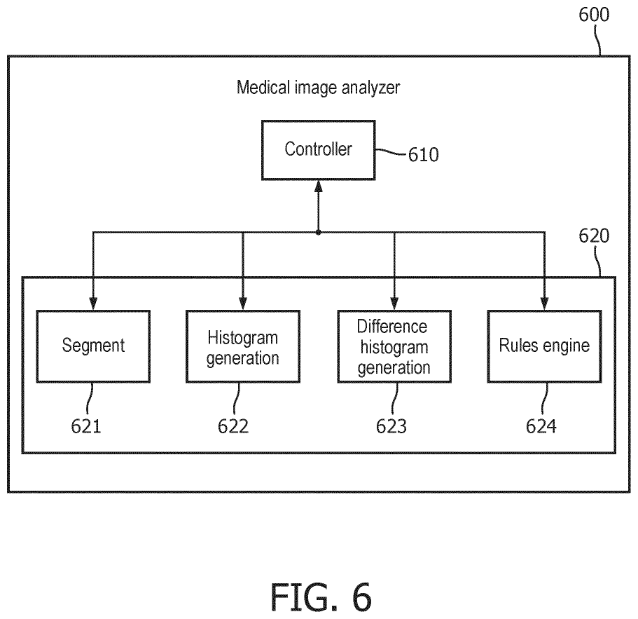

FIG. 6 illustrates an embodiment of a medical image analyzer 600 which may implement the method embodiments described herein. Referring to FIG. 6, medical image analyzer 600 includes a controller 610 and a memory 620. The controller may execute instructions stored in the memory for performing the operations and method described herein. In this embodiment, the instructions stored in memory 620 may include a first set of instructions 621 that implement a segmentation algorithm, a second set of instructions 622 that implement a histogram generator, a third set of instructions 623 that implement a difference histogram generator, and a fourth set of instructions that implement a rules engine 624. These sets of instructions may respectively perform, for example, the operations of the features in the medical image analyzer of FIG. 1 and the operations of the method embodiments described herein. In this embodiment, the segmenter and labeler may be included in the medical image analyzer. However, the segmenter and labeler may be coupled to the medical image analyzer in some embodiments, for example, as illustrated in FIG. 1.

In accordance with one or more of the aforementioned embodiments, the methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods described herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the operations of the system and method embodiments described herein.

The processors, systems, controllers, segmenters, generators, labelers, logic, engines, simulators, models, networks, scalers, and other signal-generating and signal-processing features of the embodiments described herein may be implemented in logic which, for example, may include hardware, software, or both. When implemented at least partially in hardware, the processors, systems, controllers, segmenters, logic, engines, generators, labelers, simulators, models, networks, scalers, and other signal-generating and signal-processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, systems, controllers, segmenters, logic, engines, generators, labelers, simulators, models, networks, scalers, and other signal-generating and signal-processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Although the various exemplary embodiments have been described in detail with particular reference to certain exemplary aspects thereof, it should be understood that the invention is capable of other example embodiments and its details are capable of modifications in various obvious respects. As is apparent to those skilled in the art, variations and modifications can be affected while remaining within the spirit and scope of the invention. The embodiments may be combined to form additional embodiments. Accordingly, the foregoing disclosure, description, and figures are for illustrative purposes only and do not in any way limit the invention, which is defined by the claims. The embodiments may be combined to form additional embodiments.

We claim:

1. A method for processing medical image information, comprising:
generating a first intensity distribution for a first image region;
generating a second intensity distribution for a second image region;
calculating values based on the first and second intensity distributions; and
automatically determining a custom viewing window based on the calculated values,
wherein the custom viewing window is determined to display the first image region and the second image region.

2. The method of claim 1, wherein determining the custom viewing window includes determining one or more parameters of the custom viewing window.

3. The method of claim 2, wherein determining the one or more parameters includes computing the one or more parameters based on difference values calculated between the first and second intensity distributions.

4. The method of claim 3, wherein determining the one or more parameters includes applying at least one rule to the difference values to determine the one or more parameters.

5. The method of claim 3, wherein the one or more parameters of the custom viewing window increase recognition of differences between the first and second image regions over a viewing window used to display the first and second image regions using one or more other parameters.

6. The method of claim 3, wherein the one or more parameters includes at least one of a width or a level of the custom viewing window.

7. The method of claim 6, wherein the width is based upon a maximum intensity value in the difference values for which the difference value is non-zero and a minimum intensity value in the difference values for which the difference value is non-zero.

8. The method of claim 1, wherein the values calculated based on the first and second intensity distributions include difference values between the first and second intensity distributions.

9. The method of claim 1, wherein the first image region and the second image region are in a same image.

10. The method of claim 1, wherein:
the first image region is a first area in a first brain scan image;
the second image region is a second area in the first brain scan image.

11. The method of claim 10, wherein:
the first image region is derived from a first bounding box; and
the second image region is derived from a second bounding box.

12. The method of claim 10, further comprising:
automatically determining the first and second image regions based on a segmentation algorithm.

13. The method of claim 10, further comprising:
automatically determining the second image region based on a designation of the first image region.

14. A medical image analyzer, comprising:
a generator configured to generate a first intensity distribution for a first image region and a second intensity distribution for a second image region;
computing logic configured to calculate values based on the first intensity distribution and the second intensity distribution; and
a rules engine configured to automatically determine a custom viewing window based on the calculated values, wherein the custom viewing window is determined to display the first image region and the second image region.

15. The medical image analyzer of claim 14, wherein the rules engine is configured to determine one or more parameters of the custom viewing window.

16. The medical image analyzer of claim 15, wherein determining the one or more parameters includes computing the one or more parameters based on difference values calculated between the first and second intensity distributions.

17. The medical image analyzer of claim 16, wherein the rules engine is configured to apply at least one rule to the difference values to determine the one or more parameters.

18. The medical image analyzer of claim 16, wherein the one or more parameters includes at least one of a width or a level of the custom viewing window.

19. The medical image analyzer of claim 18, wherein the width is based upon a maximum intensity value in the difference values, a minimum intensity value in the difference values, and a scalar.

20. The medical image analyzer of claim 14, wherein the first image region and the second image region are in a same image.
